(19) United States
(12) Patent Application Publication
(10) Pub. No.: US 2002/0182739 A1
(43) Pub. Date: Dec. 5, 2002

(54) Rapid detection of aromas using integrated gas chromatography with multiarray sensors

(76) Inventors: Omowunmi A. Sadik, Vestal, NY (US); Marc Breimer, Johnson City, NY (US); Miriam Masila, Endicott, NY (US)

Correspondence Address: SALZMAN & LEVY, Press Building, Press Building - Suite 902, 19 Chenango Street, Binghamton, NY, 13901, US

(21) Appl. No.: 09/891,815

(22) Filed: Jun. 26, 2001

Related U.S. Application Data
(63) Continuation of application No. 09/287,278, filed on Apr. 7, 1999, now abandoned.

(51) Int. Cl. G01N 30/00; G01N 30/74; G01N 33/00

(52) U.S. Cl. 436/106; 436/119; 436/128; 436/161; 436/164; 436/172; 422/83; 422/86; 422/89; 422/91

(57) ABSTRACT

Methods and devices are described for rapidly and simultaneously detecting, quantifying, and imaging gases, odors, malodors, volatiles and semi-volatiles using gas chromatography coupled with arrays of organic conducting polymers. The methods utilize changes in the polymer conductivity as a function of temperature to detect volatile organic molecules upon adsorption to the polymers. By raising the sensor temperature, adsorbed materials are further desorbed. This eliminates the fundamental problems of sensor fouling resulting from exposure to sulfur, nitrogen or ketone containing malodors. In a second embodiment, the resulting gas effluents from the sensor array are mixed with air, oxygen or hydrogen to produce a cloud of luminance that is detected by a charge-coupled device.

### U.S. GOVERNMENT RIGHTS

The United States Government has certain rights in this invention pursuant to U.S. EPA Grant No. R825323, given to the State University of New York at Binghamton.

### FIELD OF THE INVENTION

The present invention relates to rapid, simple, and reliable techniques and apparatuses for detecting, quantifying, and imaging gases, odors, malodors, volatiles and semi-volatile organic chemicals in a given sample using gas chromatography coupled with arrays of organic conducting polymers and, more particularly, to methods and apparatuses for detecting the presence and/or quantity of organic vapors of various aromas having different molecular weights.

### BACKGROUND OF THE INVENTION

Odor analysis is a very difficult and challenging problem. Conventional odor analysis techniques involve the use of either human sensory panels or gas chromatography (GC) combined with mass spectrometry (MS). The GC-based techniques have been widely used to conduct qualitative and quantitative analysis of odors and semi-volatile compounds. However, these methods are generally unable to reveal the true representation of odor-active components.

The use of human sensory panels for the characterization of odors and tastes is very accurate, but the time and costs involved in training each member of the panel are significant. In addition, human detection thresholds vary, depending on the individual and the compound to be analyzed. In addition, the results may be subjective. Such a technique is also unsuitable for analyzing toxic compounds. Therefore, the complexities of naturally occurring odors, their molecular instability over time, as well as the dynamics of chemical reactions at the molecular level, have made odor analysis a great challenge.

More recently, an instrumental approach has been developed which mimics mammalian olfactory systems. The system is commonly referred to as an “Electronic Nose.” The instrument consists of a combination of headspace sampling, gas sensor arrays, and pattern recognition modules that generate signal patterns used for odor characterization. The new Electronic Nose provides rapid odor analysis and reduces the subjectivity of human sensory panels. However, there exists a number of limitations that hinder the analytical application of this system for complete odor analysis. For example, Electronic Nose systems are subject to various problems, such as noise, short-term sensor drifts, problems in sampling and generating the odorous gases, and problems of mass transference at the sensor-odor interface.

Some olfactory thresholds are extremely low. Certain compounds having vapor pressures, too low to register in many Electronic Nose profiles, nonetheless are important odorants. Therefore, odors (or volatiles) measured by sensor techniques, and the concentration of essential odorants, are not necessarily correlated. Moreover, headspace sampling may lead to poor signal reproducibility which may permanently and significantly alter the bulk (mole number) of the system. This has the effect of removing subsequent samples, which leads to inconsistent results. This problem is more serious for small volumes and organic solutes having very low solubility (e.g., aliphatic and aromatic hydrocarbons).

The Electronic Nose odor analysis provides chemical description in terms of the sensitivity limits of the gas sensor instrument and the sample preparation technique. Therefore, what is required is an alternative (e.g., hyphenated) technique capable of defining sensor-odor activity relationships in terms of the whole analytical process, not merely the sensor array.

The present invention introduces the concept of a quantitative, sensor-odor activity relationship to improve Electronic Nose performance for analyzing volatiles. This involves: i) separating complex odors using a column, ii) detecting the complex odors using an array of sensing elements made of organic conducting polymers, iii) imaging the complex odors using a charge-coupled device, iv) linking the chemical image or profiles acquired with library data bank, and v) comparing the image or profiles with the data bank to extract quantitative information about the odor. This hybrid sensor, known as a Gas Chromatography-based Electronic Nose (GC-EN), results in very rapid analysis as well as improved sensitivity and selectivity. The system consists of a gas chromatograph and sensor array instrument capable of analyzing complex gases, or vapors, for odors and semi-volatiles.

The GC-EN consists of a commercially available sensor array system and gas chromatograph, capillary column, and electronic switch-box. The column effluents are made to pass through dual flame ionization and sensor array detectors with the output recorded by a signal processor. The carrier gas is allowed to flow continually from the column through the detectors, thus allowing the detectors to be selected for odor identification. The odor components are then separated by the column using their relative retention times and vapor pressures.

The electronic switch-box is used to control effluent temperature prior to introduction into the sensor chamber and to clean the sensors of any adsorbed materials. This works by selecting the inlet block temperature in certain steps, from column temperature (as high as 280° C. to ambient), while the sensor array is being applied to identify the odors. The temperature control is important to prevent the sample from getting too hot and decomposing or too cold and condensing. The results from the sensors are fed into pattern recognition modules, where further identification of the vapors is accomplished. Both the information extracted from the chromatographic separation and the sensors are combined to generate a complete, quantitative odor-activity relationship.

### SUMMARY OF THE INVENTION

The present invention provides for a compact odor analyzer apparatus and method consisting of a sensor chamber comprised of gas-phase array (GPA) devices coupled to a chromatographic module. A typical configuration, in accordance with this invention, consists of a compact GC (gas chromatographic) system equipped with an electronic switch-box and a polymer-modified array device. These GPA devices are commonly referred to as multi-array detectors used for assaying the presence, the quantity, and the chemical profiles of odors. The gas phase arrays have a chemically-selective layer and a charge-coupled camera. The chemically-selective layer is used for separating a particular odor, while the charge-coupled camera is used in the detection of luminance intensity of the odor sample. The method associated with the apparatus, comprises the steps of:

a) injecting the sample through an analysis system which separates and analyzes the sample into its active components;

b) contacting the sample with the GPA containing a series of custom-made polymer CCD detector arrays for detecting the luminance intensity of the sample as well as the chemical profile; and

c) modulating the selective interaction of the sample with the polymers in a sensitive and predictable manner, which exhibits high cross-reactivity for the maximum number of components being determined. This sensitive analysis provides pertinent information about the nature, the type, and the level of the odor.

In a second embodiment, a typical gas chromatograph is linked to the coupling device through a charge-coupled camera. The quantitative and qualitative separation of individual gas components defines the composition of the sample, and the effluents from the chromatograph are then exposed to the GPA. The combination of gas chromatography with sensor array coupled with charge-coupled device allows separation of odor components into individual molecules, simultaneously detect multiple analytes in complex samples and image the odorants.

The characteristic fingerprint of the sample is obtained from a library of molecular properties of odors, which is used to compare the properties of one odor molecule to another. A complete chemical image, associated with the analyte of interest, is determined by linking the acquired chemical image with that of the complex profiles obtained from the library data bank.

The present invention is suitable for odor characterization in several different applications including, but not limited to: a) simple, fast, and reliable screening of environmental compounds; b) assessing odors for forensic and law enforcement applications; c) monitoring odors associated with certain diseases; d) classifying packaging and paperboards; e) detecting associated odors in plastic manufacturing materials; f) validating cosmetics and toiletries; g) determining food freshness and herbicides used in agricultural products; and h) surveillance of chemical agents of mass destruction and biological warfare.

The advantages of the invention, include:

Rapid results for environmental surveillance programs

Compact system, amenable to field deployment

Cheaper alternatives to existing laboratory methods

Low-cost, qualitative and quantitative odor analysis

On-line and reproducible sample introduction for odor analysis

Rapid odor analysis within minutes, as compared to several hours in existing techniques

Easy integration to current instrumental methods.

It is an object of this invention to provide improved apparatuses and methods for detecting and analyzing odors.

It is another object of the invention to provide methods and apparatuses for detecting and analyzing a wide range of odors that were heretofore unanalyzable by other techniques.

It is a further object of this invention to provide methods and apparatuses for detecting low fraction volatiles and odors.

### BRIEF DESCRIPTION OF THE DRAWINGS

A complete understanding of the present invention may be obtained by reference to the accompanying drawings, when considered in conjunction with the subsequent detailed description, in which:

FIG. 1a is a schematic diagram of the system in accordance with the present invention.

FIG. 1b is a simplified block diagram of the layout of the Gas Chromatograph-coupled Multi-array Sensors and Thermal Energy Transfer Mechanism of the invention;

FIG. 2 depicts a schematic diagram of the sensor chamber showing the heat transfer therein;

FIG. 3 shows a schematic of the sensor array and TEC controller and electronics;

FIGS. 4a and 4b respectively illustrate block diagrams of the multichannel sensor array design with the unique polymer film sensing;

FIG. 5 depicts the flow routing of the sample, utilizing the inventive system;

FIG. 6a shows a block diagram of a first inventive testing scheme;

FIG. 6b illustrates a side view of the split injector coupling used for coupling gas chromatography with the multi-array sensors;

FIG. 7 shows a block diagram of a second inventive testing scheme;

FIG. 8 depicts a block diagram of a third inventive testing scheme;

FIG. 9 illustrates a chromatogram of GC-EN responses for 32-sensor arrays, using polyaromatic hydrocarbons as the analytes;

FIG. 10a depicts the chromatogram of a 2 μL injection of octane in the GC-EN system;

FIG. 10b shows the chromatographic response to m-xylene for the GC-EN system;

FIG. 10c illustrates the chromatographic response of 2 μL injections of decane into the GC-EN system;

FIG. 11 depicts a chromatographic response to successive injections of a 2 μL mixture of the octane, m-xylene and decane;

FIG. 12 shows a graphic response obtained for a series of methanol injections using the Electronic Nose without any column;

FIG. 13 illustrates a plot of the normalized sensor response for repetitive injections of 1.0 μL acetone;

FIGS. 14a, 14b and 14c respectively depict the chromatographic response for the GC-EN separation of 3-CP and toluene;

FIGS. 15a and 15b respectively show a graph of the retention times and the areas obtained for low molecular weight solvents using the GC-EN system;

FIG. 16 graphically illustrates the linear responses obtained for all of the low-molecular weight analytes studied, with their correlation coefficients; and

FIG. 17 graphically depicts the results obtained for the total recognition of 2,4-dichlorophenol (2,4-D) using an artificial neural network.

### DESCRIPTION OF THE PREFERRED EMBODIMENT

Generally speaking, the invention features methods and devices for rapidly detecting, quantifying, and imaging volatile and semi-volatile odors using gas chromatography coupled with polymer-modified multi-array sensors. The method utilizes the concept of quantitative, sensor-odor activity relationships to improve the analytical performance of the multi-array sensor in its analysis of odor components.

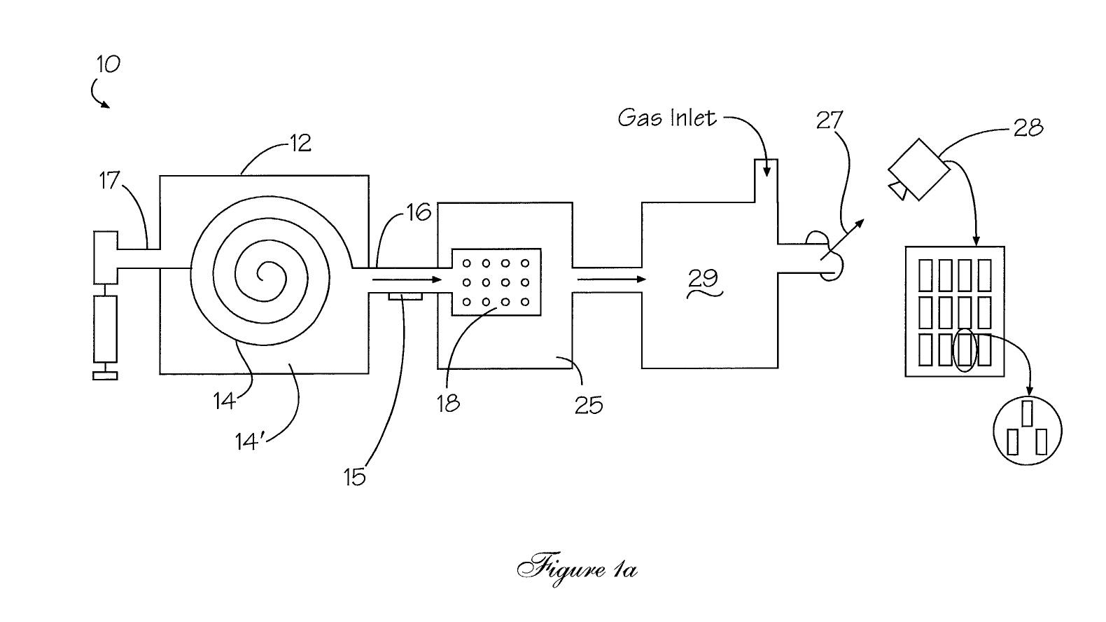

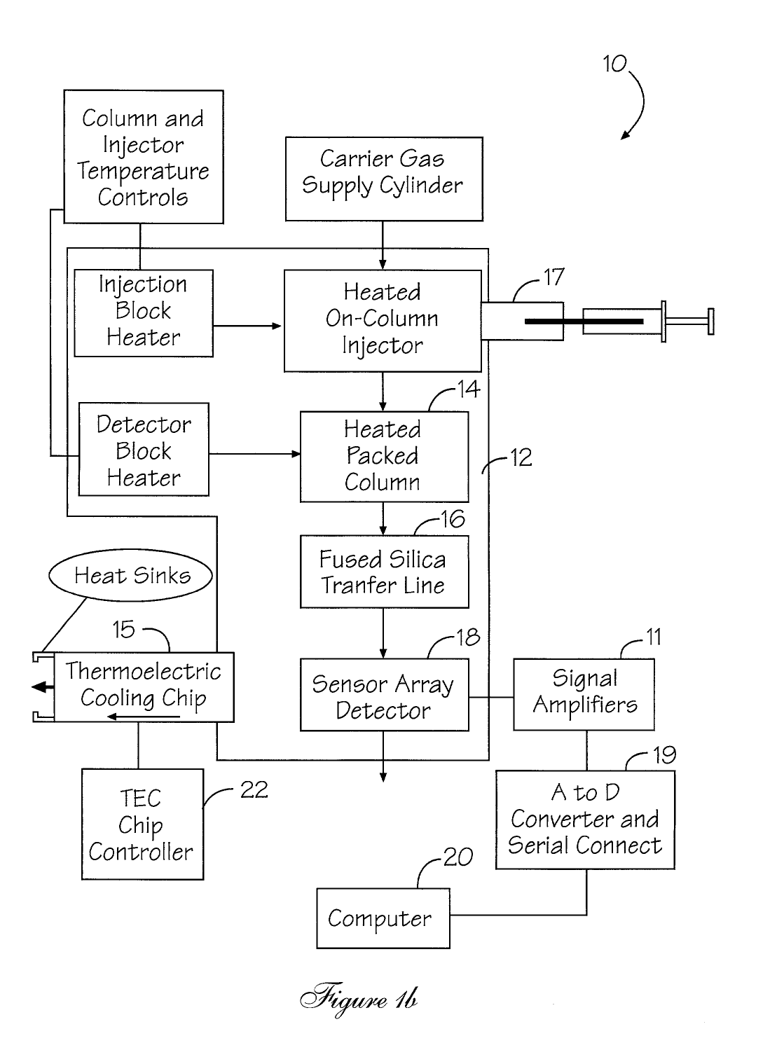

Referring now to FIGURES 1a and 1b, a system 10 is schematically illustrated, which comprises a gas chromatograph (GC) 12 equipped with either a capillary or a packed column 14 that separates the odor components that are injected into the system at reference numeral 17. The other components of system 10 include a fused silica transfer line 16, sensor array detector 18, computer 20, Thermoelectric Cooling Chip 15, and Thermoelectric Cooling Chip controller 22. Connected to sensor array 18 in sensor chamber 25 is a mixing chamber 29. A luminance cloud 27 is formed downstream of mixing chamber 29. A charge-coupled device (CCD) 28 is operatively positioned relative to the luminance cloud 27 to detect effluent as described hereinbelow.

The fused silica transfer line 16 carries the eluting gas mixture from the GC 12 into the sensor array detector 18. The computer 20 analyzes the odor components present in the sensor array detector 18 via an A/D converter 19 and signal amplifiers 11.

The thermoelectric cooling (TEC) chip 15 and TEC controller 22 control the effluent temperatures in the sensor array detector 18 through a differential mechanism. The three thermal energy transfer mechanisms of the sensor array 18 are shown in FIG. 2. Heat is transferred by conduction a) through the oven 14′ to the enclosure wall interface; b) through the injector 17 to the enclosure wall interface; and c) through the fused silica capillary wall 16, as shown by arrows 30.

The effluent is cooled and fed into sensor chamber 25 of the sensor array detector 18, where the gas components are identified. Thereafter, the results from the detectors 18 are fed into a pattern recognition module 26 (FIG. 4a), where the signal is processed. Both the information extracted from the chromatographic separation and that of the sensor identification processes are combined to give a complete, quantitative odor analysis.

The sensor chamber 25 is made up of a supporting member and an array of chemically selective polymer layers formed from heterogeneous and quasi-selective thin films. These films act as the sensing receptor unit, and are able to detect a variety of analytes using pattern recognition protocols, as illustrated in FIG. 3. The supporting members can be microelectrode arrays, quartz crystal microbalance, or optical sensors. In any case, the supporting members may be individually assessed using a potentiostat as a constant potential source (FIGS. 4a and 4b) and a charge-coupled device for imaging the sensor output.

The charge-coupled device is used for imaging. After the analytes have been separated by the column and detected by the sensor array, if malodor components are present, they cannot be detected by the sensor array. In fact, these components are known to foul the sensing array. In this case, the polymer arrays are heated to desorb these malodor components and the resulting effluents are mixed with specialty gases, generating luminescence. It is the luminescence that is imaged by the charge-coupled device.

This new method can also be used as a gas-phase homolog of a flow injection analysis, which is typically used for liquid-phase systems, where the separation mode is excluded and the analysis carried out simply by injecting the analytes through the mobile gas sensors, as illustrated by Route 2 of FIG. 5. In this mode, detection is also achieved using the multiarray.

The device works on the basis of changes in surface conductivity of the polymer arrays upon exposure to the odors. By controlling the temperature of the effluent coming from the column and the sensor array using the electronic switch box, the conductivity of the polymers for different volatile molecules can be modulated. The separation is carried out in a column, using chromatography to separate the individual components of an odorant sample by using the differences in their retention time. The luminescent and the imaging components are only useful for analyzing malodors. Typically malodors are the result of a sample containing either sulfur, nitrogen or ketone functional groups. These functional groups will cause a fouling of the sensor array and may therefore elude detection. Consequently, the effluent is further reacted with specialty gases (i.e., air, oxygen or hydrogen) out of the sensor chamber as seen in the following examples:

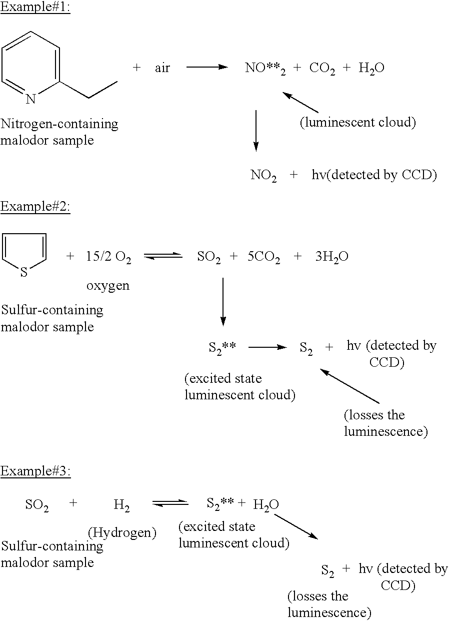

The resulting luminescent produced from these reactions are detected by the charge-coupled device (CCD). This is the imaging component of the invention.

In addition, this new system is capable of detecting a wide range of volatile and semi-volatile organic compounds in a given sample. Such analytes include, but are not limited to, those listed in Table 1, below. The system has been used to analyze several organics, including aromatic hydrocarbons, halogenated organics, and volatile industrial chemicals. The procedure employed is explained hereinafter.

TABLE 1
Examples of Detectable Analytes Using the Inventive System

| | |
|---|---|
| Hydrocarbons | alkanes, and alkynes ranging from |
| | 1-30 carbon atoms, anthracene, |
| | pyrene, benz(a)pyrene, toluene, |
| | ethers, ketones, alcohols, |
| | nitriles, hydrazine, cocaine and |
| | metabolites, acetic esters |
| Halogenated organics | phenols, chlorophenols, |
| | pentachorophenol, 2,4 |
| | dichlorophenols, microbial phenol |
| | degradation |
| Odors of spices | Sesquiterpene alcohol |
| | d-camphene, beta-phellandrene, |
| | citral, decylic aldehyde, diallyll |
| | disulfide, galic, allyl propyl |
| | disulfide, higher sulfides |
| Organic Solvents | Acetone, benzene, ethylacetate, |
| | xylenes, toluene, |
| | tetrachioromethane, |
| | trinitritoluene, ethylbenzene |
| Environmental compounds | Methane, formic acid, ammonia, |
| | Benzoate, gasoline vapors, toluene, |
| | n-butyl acetate, triethylamine |
| Odorous Products | Cocoa, coffee, pyridines, maltol, |
| | thiazole |

Procedures:

A commercially available gas chromatograph, a capillary column, an electronic switch-box, and a commercially available sensor array system were used in the inventive process. The column effluents were made to pass through dual flame ionization and sensor array detectors, where the output was recorded by the signal processor. The carrier gas flowed continually from the column into the detectors, thus allowing each detector to be selected for odor identification. The column separated the odor components according to their relative retention times and vapor pressures. An electronic switch-box was used to control the effluent temperature prior to introduction into the sensor chamber. This was accomplished by selecting the inlet block temperature in certain steps, from the column temperature which is as high as 280° C., to ambient temperature.

Then, the sensor array was used to identify the odors. The results from the sensors were fed into a pattern recognition module, where further identification of the vapors was achieved. Both the information extracted from chromatographic separation and that extracted from the sensors were combined to generate a complete quantitative odor-activity relationship.

In this novel GC-EN technique, several parameters were used to define odorous samples. These include peak shapes, relative retention times, peak areas, relative slicing time, adsorption/desorption kinetics between sensor, and odor molecules.

The types of sensing polymers used in the procedure are shown below, in Table 2.

TABLE 2
Examples of Sensing Polymers Used in Multiarray Sensors

Polypyrrole/Polyethylene Oxides
Polystyrene/Polypyrrole
Polypyrrole/polyvinyl chlorides
Polythiophenes
Polyurethanes/polypyrrole
Polyaniline
Polypyrrole

Three novel schemes were employed to integrate the gas chromatographs with the sensors. These schemes are illustrated in FIGS. 6a, 6b, 7, and 8, respectively. Scheme 1, shown in FIG. 6a, utilized a split injector 32 for the capillary columns to introduce sample 31 to the multiarray sensors 34. The GC effluent could also have been channeled to a standard flame ionization detector 37. The GC effluent 36 was channeled to the inlet of the multiarray sensor 34 using a 15 inch long, 0.762 nm internal diameter and 1/16 inch external diameter tubing.

The GC end was run through the sensor block, into the oven 14′, where a Swagelock 1/16 inch nut ferrule kit was attached and subsequently connected to a 1/16 male-to-male union, as shown in FIG. 6b. An Upchurch Finger-tight, flanged fitting was connected to the other end of the tube. This fitting was attached to a union, which connected it to a male Luer lock. This Luer lock fitting was used to couple the tube to the sampling port of the sensor array through stainless steel tubing. The flow rate was approximately 20 ml/minute at a pressure of 30 psi. A small portion of the sample went to GC column 36. Typical split ratios employed ranged from 70:1 to 200:1.

In Scheme 2, the column effluent was fed into the multiarray sensor 34. Similar benefits were provided as in Scheme 1. Furthermore, this scheme allowed the separation of volatile components contained in the sample to be detected by the thirty-two sensor arrays of this system. In addition, this scheme is useful for the identification of odorant molecules in the materials.

In Scheme 3, similar benefits as those of Schemes 1 and 2 were provided, but this scheme was particularly suitable for analyzing high volatile compounds where large temperature variation between the GC 36 and the sensor system 34 led to excessive peak broadening.

Preparation of Sensor Arrays:

Sensor arrays were modified with different polymers using electrochemical polymerization techniques. Polymer sensor arrays were fabricated by electrochemical polymerization (O. A. Sadik, G. G. Wallace, Electroanalysis, 5 (1993) 555-563, Bender S., 0. A. Sadik, Environmental Science & Technology, 1998, 32, 788-797). Interdigitated gold electrodes were first precleaned using “Piranha Solution” (H₂SO₄:H₂O₂=3.1). The conducting polymer layers were fabricated by applying a constant current of 1 mA. The monomer solution was prepared by dissolving 0.1-0.5 M pyrrole in deionized water (or acetonitrile) which also contained 0.1 M of a supporting electrolyte such as NaNO₃, NaCl, sodium dodecylsulphate, or tetrabutylammonium perchlorate. The instrument for the electrochemical polymerization was EG & G, PAR potentiostat/galvanostal Model 263. The auxiliary electrode was platinum gauze and the reference electrode was silver-silver chloride. Polymerization was maintained until the gap (ca. 16 μm) between two metal lines was filled with the polymer and this approach produced sensors with discrete resistance.

Applications:

The GC-EN system of the present invention has been evaluated for the analysis of a range of model analytes including industrial solvents, polyaromatic hydrocarbons, and chlorinated phenols. Other analytes include 2,4-Dichlophenols, 2-Chlorophenols, Benzo-a-pyrene, 9,10-Dihydrophenanthrene, Benzanthracene, Benzo(a)pyrene, Xylenes, Toluene, 3-Chlorophenol, Acetone, and Hexane. These compounds were chosen due to their relative molecular weights, boiling points, nature and type of functional groups, toxicity, and industrial significance.

Example 1: Analysis of Polyaromatic Hydrocarbons

Referring to FIG. 9, the response of the GC-EN having an array of 32 sensors, using polyaromatic hydrocarbons as the analytes, is illustrated. The results indicate that the separation of benzo-a-pyrene and pyrene were achieved in less than five minutes. The intensities of different sensors to the analytes were clearly distinguished based on the percentage change in sensor resistance. Well-defined responses were recorded for pyrene at all of the 32 sensors; whereas for benzo-a-pyrene, only sensor numbers 5, 6, 9, 15 and 21 produced the most intense signals. Other sensors produced relatively wide responses. Sensor numbers 1, 12, 18, 28, and 32 were completely flat, thus signifying that these sensors were not selective for benzo-a-pyrene.

Example 2: Analysis of Semi-volatile Organic Compounds

The GC-EN system was also used to analyze semi-volatile organic compounds, including octane, m-xylene, and decane. These compounds were used to demonstrate the applicability of the structure-activity relationships discussed above. These compounds were also based on the influence of analyte parameters such as the chain lengths and boiling points.

The chromatogram of a 2 μL injection of octane is shown in FIG. 10a. The graph of the percentage change in sensor resistance (percentageΔR/R x 100) versus the sampling time produced resulted in peaks for the highly responding sensors. The most sensitive peaks for octane were recorded for sensor number 17, having a retention time of 5.4 minutes. Other sensors responding highly were numbers 2, 9, 10, 17, 18, 20, 22, 23 and 24.

FIG. 10b shows the response to m-xylene of the GC-EN. It was observed that similar sets of sensors produced the most sensitive peaks, although the peaks became broader after about 6.25 minutes. With 2 μL injections of decane, sensors number 17, 18, 20, 22, 23 and 24 produced the highest responses with retention times of 8.25 minutes, as illustrated in FIGURE 10c.

Successive injections of the 2 μL mixture of the octane, m-xylene and decane were conducted. The results shown in FIG. 11 confirmed that the retention times for the three analytes were 5.4, 6.25 and 8.3 minutes, respectively, as shown in FIG. 11. Sensors number 2, 9, 10, 17, 18, 20, 22, 23 and 24 responded to the octane and m-xylene, whereas sensors number 2, 9, and 10 produced a very broad peak for decane. Therefore, these were not sensitive to decane. The shapes of the peaks can be used to provide an indication of the mechanism of the odor interaction with the sensors. The different shapes of the peaks can be explained in terms of the adsorption/desorption kinetics between each analyte and the sensor arrays. Since different analytes have different adsorption/desorption kinetics, the change in resistance for the sensor is expected to be different.

Referring now to FIG. 12, illustrated is the response obtained for a series of methanol injections using the Electronic Nose without any column. The response was much sharper and reproducible when different volumes of samples were injected at standard deviation of less than 0.1%. Therefore, the analysis of volatile without any separation column also suggests that by simply changing the sample introduction method in standard EN systems, the sensitivity improves by several orders of magnitude. The reproducibility of the technique was tested for all of the analytes investigated. In all of the cases, the greatest standard deviation obtained was less than 2%.

Referring now to FIG. 13, a plot is shown of the normalized sensor response for repetitive injections of 1.0 μL acetone. Reproducible sample volumes were recorded for all of the sensors.

Example 3: Analysis of Industrial Organic Solvents and Chlorinated Hydrocarbons

The GC-EN system of the present invention was also used to analyze common solvents. These include acetone, toluene, hexane, and chlorinated organics such as 2,4-dichlorophenol (2,4-D), 3-chlorophenols, 2-chlorophenols and toluene. A 2:1 mixture of toluene and 3-chlorophenol (3-CP) was prepared by adding 2.00 ml of toluene and 0.990 ml of 2-CP proportions by carbon mass. Another mixture containing acetone, toluene and hexane was prepared to yield a final solution having equal carbon mass-weighted proportion of each solvent. The mixtures were injected into the GC column and the chromatograms recorded. The GC-EN separation of 3-CP and toluene is shown in FIG. 14. These chromatograms are the signatures recorded for the injection of toluene, 3-CP and other mixtures, as well as the average sensor response for the analytes.

These results were compared with those recorded for a standard GC-FID (flame ionization detector) system. The retention times and the areas obtained for low molecular weight solvents are shown in FIGS. 15a and 15b, respectively. These results confirm that the retention times and the peak areas depend on the hydrocarbon chain lengths. The inventive GC-EN system produced comparable results with a conventional GC using flame ionization detectors 37 (FIG. 6a), except that slow dynamics in the sensor housing adversely affected the appearance of the peaks.

Good linear responses were obtained for all of the low-molecular weight analytes studied, with correlation coefficients in the range 0.9598 to 0.9902, as depicted in FIG. 16. Overall, the EN detector showed that the sensor response was affected by the chemical identity of the eluting species. This implies that the EN is capable of identifying analytes eluting from the GC, and therefore satisfies the requirements as a detector.

Example 4: Analysis of Phenol and Metabolites

Industrial discharge of phenol and its derivatives often causes the contamination of ground water, resulting in odor thresholds in the ppb (samples g/L) range. When present in the ppm range, they make ground water completely unsuitable for drinking or cooking. Odors consisting of 2, 2,4-, and 2,6 chlorinated phenols are among the products of electrophilic aromatic substitution of phenols with extremely penetrating “antiseptic” odor characteristics.

The GC-EN system of the present invention was also used for the analysis of phenols having a different number of substituent groups. Previous work has shown that there is a direct relationship between the nature of the substituents, types and the molecular weights. This is likely to be the essential factor in determining the strengths of the odor-sensor interactions. Further structure-activity relationships using conducting polymer interface showed that the most toxic members of the phenolic family were those with the highest sensor intensities.

Referring now to FIG. 17, the results obtained for the total recognition of 2,4-dichlorophenol (2,4-D), using artificial neural network, is illustrated. The result confirms that 2,4-D could be easily recognized.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

1. A gas chromatography-based electronic nose (GC-EN) apparatus for analyzing gases and odors, comprising:
an electronic coupler for controlling the effluent temperature prior to sample contact with the sensor chamber; and
gas-phase array devices coupled to said chromatographic column for receiving and detecting said odor sample and for assaying the presence, the quantity, and the chemical profiles of odors, said gas-phase array devices each having a chemically-selective layer for separating a particular odor in the odor sample.

2. The gas chromatography-based electronic nose (GC-EN) apparatus for analyzing gases and odors in accordance with claim 1, wherein said array comprises electronic devices.

3. The gas chromatography-based electronic nose (GC-EN) apparatus for analyzing gases and odors in accordance with claim 1, wherein said array comprises means for obtaining optical measurements.

4. The gas chromatography-based electronic nose (GC-EN) apparatus for analyzing gases and odors in accordance with claim 1, wherein said array comprises means for obtaining acoustic measurements.

5. The method of analyzing an odor sample, comprising the steps of:
a) injecting an odor sample into an analyzing system
b) introducing the sample to a series of polymer detector arrays for receiving and selectively detecting each odor of said odor sample and further for assaying its presence, quantity, and chemical profile in said odor sample; and
c) selectively detecting each odor of said odor sample within the polymer detector arrays to provide a sensitive analysis of the nature, type and level of each odor in said odor sample.

6. The method of analyzing an odor sample in accordance with claim 5, further comprising the steps of:
d) linking a chemical image acquired with that of complex profiles obtained from a library data bank; and
e) comparing said chemical image with data in said library data bank.

7. A chromatographic apparatus for analyzing gases and odors, comprising:
a chromatographic coupler for receiving an odor sample;
gas-phase array devices coupled to said chromatographic coupler for receiving and detecting said odor sample and for assaying the presence, the quantity, and the chemical profiles of odors, said gas-phase array devices each having a chemically-selective layer for separating a particular odor in the odor sample; and
means for linking said chromatographic coupler to and said gas-phase array through a charge-coupled camera for imaging said odors in said odor sample.

8. The chromatographic apparatus for analyzing gases and odors in accordance with claim 7, wherein said array comprises electronic devices.

9. The chromatographic apparatus for analyzing gases and odors in accordance with claim 7, wherein said array comprises means for obtaining optical measurements.

10. The chromatographic apparatus for analyzing gases and odors in accordance with claim 7, wherein said array comprises means for obtaining acoustic measurements.

11. The chromatographic apparatus for analyzing gases and odors in accordance with claim 7, further comprising means defining a library of molecular properties of odors, and comparing means linked to said library and said detector array for comparing the properties of one odor molecule to another.

12. The chromatographic apparatus for analyzing gases and odors in accordance with claim 11, wherein said means defining a library of molecular properties of odors further comprises a fingerprint of each odor being analyzed.

13. A gas chromatography-based electronic nose (GC-EN) apparatus for analyzing gases, odors, malodors, volatiles, and semi-volatiles, comprising:
a) a chromatographic column for receiving an odor sample to be analyzed; and
b) a sensor chamber comprising a plurality of gas-phase array devices coupled to said chromatographic column for receiving and detecting effluent of said chromatographic column containing said odor sample, and for assaying the presence, the quantity, and the chemical profiles of odors, said gas phase array devices each having a is chemically-selective layer for separating a particular odor in said odor sample and further comprising a charge-coupled camera for detecting the intensity of luminance of said odor sample.

14. The GC-EN apparatus in accordance with claim 13, further comprising:
c) a temperature controller operatively connected to said chromatographic column for controlling the temperature of said effluent prior to odor sample contact with said sensor chamber in order to prevent condensation or decomposition of said odor sample.

15. The GC-EN apparatus in accordance with claim 14, further comprising:
d) a library data bank comprising data with which said chemical profiles of said odor sample are compared.

16. A method of analyzing odor samples, comprising the steps of:
a) injecting odor samples into an analyzing system substantially simultaneously, said analyzing system separating and analyzing said samples into the active components thereof;
b) reacting effluents from a sensor with specialty gases, resulting in a luminescence cloud;
c) introducing the samples to a series of polymer CCD detector arrays for receiving and selectively imaging the intensity of the luminance of each odor of said odor samples and further for assaying the presence, quantity, and chemical profile of each of said odors in said odor samples;
d) selectively detecting the intensity of the luminance of each odor of said odor samples within said polymer CCD detector arrays to provide a sensitive analysis of the nature, type and level of each odor in said malodor samples; and
e) controlling polymer-conductivity through heating, thereby releasing adsorbed fouling components and preventing fouling of polymeric sensing layers upon exposure to said malodor samples.

17. The method of analyzing odor samples in accordance with claim 16, further comprising the steps of:
f) linking a chemical image based on said luminance intensity detection with that of complex profiles obtained from a library data bank; and
g) comparing said chemical image with data in said library data bank.

18. The method of analyzing odor samples in accordance with claim 16, wherein said method results in analyzing low molecular weight malodor compounds using the presence of nitrogen, sulfur and ketone- containing functional groups.

19. A chromatographic apparatus for analyzing gases and odors, comprising:
a charge-coupled camera for imaging odors contained in an odor sample by detecting the intensity of luminance of said odor sample;
a gas chromatograph for receiving an odor sample to be analyzed;
gas-phase array sensing chambers coupled to said gas chromatograph for receiving and detecting said odor sample, and for assaying the presence, the quantity, and the chemical profiles of odors, said gas-phase array sensing chambers each having a chemically-selective layer for separating a particular odor in the odor sample; and
means for linking said gas chromatograph and said gas-phase array devices to a charge-coupled camera for imaging said odors in said odor sample by detecting the intensity of luminance thereof.

20. The chromatographic apparatus for analyzing gases and odors in accordance with claim 19, further comprising means defining a library of molecular properties of odors, and comparing means linked to said library and said detector array for comparing the properties of one odor molecule to another.

21. The chromatographic apparatus for analyzing gases and odors in accordance with claim 20, wherein said means defining a library of molecular properties of odors further comprises a fingerprint of each odor being analyzed.
